(19) United States
(12) Patent Application Publication
(10) Pub. No.: US 2026/0233030 A1
(43) Pub. Date: Aug. 13, 2026

(54) SYSTEM AND METHODS FOR COMMISSIONING IN TREATMENT PLANNING

(71) Applicant: UNM Rainforest Innovations, Albuquerque, NM (US)

(72) Inventors: Richard SHAW, Albuquerque, NM (US); Shuang LUAN, Albuquerque, NM (US)

(21) Appl. No.: 19/534,641

(22) Filed: Feb. 9, 2026

Related U.S. Application Data
(60) Provisional application No. 63/755,399, filed on Feb. 7, 2025.

(51) Int. Cl. A61N 5/10; G06N 3/08; G06N 3/084

(52) U.S. Cl. CPC A61N 5/1045; A61N 5/1031; A61N 5/1042; G06N 3/08; G06N 3/084

(57) ABSTRACT

The invention introduces a compact, lightweight neural network model capable of being deployed on mobile or embedded devices for real-time applications, enabling physicists to perform linear accelerator (LINAC) commissioning with greater simplicity and sophistication. This advancement ensures accessibility, efficiency, and precision, revolutionizing future LINAC commissioning.

### CROSS REFERENCE TO RELATED APPLICATIONS

This application claims the benefit of U.S. Provisional Patent Application No. 63/755,399, filed on Feb. 7, 2025, the contents of which are incorporated herein by reference.

### FIELD OF THE INVENTION

The invention relates generally to the field of oncology. More specifically, the invention relates to commissioning in treatment planning using a neural network model.

### BACKGROUND OF THE INVENTION

Radiation therapy is a well-established and effective modality for cancer treatment, and over half of cancer patients undergo radiation therapy. Modern radiation therapy relies on a treatment planning system (TPS) to compute high quality therapeutic plans, which are delivered using a medical linear accelerator (LINAC).

A TPS consists of two major components, an accurate radiation dose calculation engine and a powerful mathematical optimization algorithm to compute a treatment plan that maximizes tumor dose while minimizing nearby healthy tissue radiation exposure. Before clinical use, TPS must be “commissioned” to establish an accurate physics model of its intended delivery LINAC. After the initial commissioning, it is also important to establish a robust quality assurance (QA) program to maintain reliability over time.

Standard commissioning uses a heavy 300-lb water tank (50 cm×50 cm×50 cm) to measure LINAC radiation output across many different field sizes and depths with sub-millimeter precision. This cumbersome process also requires a dedicated lift for set-up, which often introduces additional errors. The commissioning process itself is labor-intensive and tedious, requiring thousands of precise measurements. FIG. 1 shows examples of the radiation dose profiles obtained from commissioning measurements, which are imported into the TPS for building a LINAC physics model.

Despite advances in automation, the price of high-quality data comes at the expenses of long working hours and significant delays in patient treatments. Studies also revealed many institutions fail to deliver radiation dose within acceptable errors, often due to commissioning data inaccuracies. Many radiation oncology clinics use vendor-provided so-called “golden beam” datasets to verify commissioning quality. However, the American Association for Physicists in Medicine (AAPM) recommends against such practice due to the need for site-specific adjustments and the inherent uncertainties in LINAC manufacturing.

Recently, there has been a surge in interest in the applying deep learning to healthcare applications, as these technologies have demonstrated state-of-the-art performance. For LINAC commissioning and QA, previous methods have proposed a multivariant regression model to reduce human errors and manpower. By correlating radiation dosimetry data with LINAC features, their model achieved low prediction errors. However, the approach requires a large number of training datasets to stabilize the model's predictive ability, posing challenges in commissioning applications (it is believed that there are only around 3,500 LINACs in the US).

Other previous methods have demonstrated the state-of-the-art results in modeling LINAC beam data using SIREN (Sinusoidal Implicit Representation Networks). They attributed the success to the sine function's oscillatory nature which makes it highly sensitive to capture high-frequency features. However, this oversensitivity also makes the model prone to capturing noise as high-frequency features, leading to overfitting and poor generalization, especially when applied to smaller neural networks that are desired for clinical deployment.

What is needed is a suitable neural network model and activation function for representing spatially variant LINAC radiation beam data, and to develop a small noise-resistant neural network for producing full physics data set needed for commissioning and QA from a few physical measurements. The invention satisfies this need.

### SUMMARY OF THE INVENTION

The two cornerstones of radiation cancer therapy are medical linear accelerators (LINACs) for producing high energy X-rays and treatment planning systems (TPS) for computing the optimal therapeutic plans. The goal is to deliver targeted lethal doses to destroy tumors while sparing surrounding normal tissues. For LINAC and TPS to work seamlessly together, TPS must have an accurate physics model of the LINAC. To do this, commissioning is performed where extensive measurements of the LINAC are made and uploaded into the TPS. This is a tedious and expensive process, taking months to complete. To ensure continuous and accurate delivery of the treatment dose, following the initial commissioning, routine quality assurance must also be performed where measurements are made and compared.

As mentioned above, commissioning LINAC for clinical use requires extensive TPS validation, typically involving large measurement datasets that demand significant time, expertise, and resources. The invention substantially reduces the volume of commissioning measurements by using a neural network model capable of generating complete commissioning datasets from only a small subset of measured profiles.

The invention provides an end-to-end commissioning technique using data-driven methods for design of an optimal detector to commission a treatment planning system, quickly. By utilizing techniques from information theory, machine learning, and pattern recognition the technology according to the invention can identify the optimal number of datasets required for commissioning. With these results, measurement protocols can then become rigidly standard with physicists focusing on the most consequential measurements. Dramatically simplifying commissioning and regular QA ensures the high-quality maintenance of clinical systems along with significant time savings for staff.

For a LINAC and treatment planning system (TPS) to work seamlessly together, the TPS must be tweaked and validated during the commissioning process. Commissioning involves taking an extensive set of measurements that require countless man-hours, expertise, and expensive equipment. The invention drastically trims the size of these data sets using a neural network model to generate all the necessary commissioning data from a fraction of the measurements.

According to the invention, the model uses a 2-stage training process, where in the first stage, the model is trained with one full data set from a similar machine; while in the second stage the model is “conditioned” and “refined” with one measurement for the intended LINAC before producing the full data set.

Since the model is deployed for clinical use and requires fast conditioning, it is important that the model be as small as possible. Also, it is noise resistant avoiding setup errors that may occur during physical measurement.

The invention and its attributes and advantages will be further understood and appreciated with reference to the detailed description below of presently contemplated embodiments, taken in conjunction with the accompanying drawings.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the presented detailed description. The description and accompanying drawings may highlight a certain specific example, or a certain aspect of the invention. However, one skilled in the art will understand that portions of the example or aspect may be used in combination with other examples or aspects of the invention.

The preferred embodiments of the invention will be described in conjunction with the appended drawings provided to illustrate and not to limit the invention.

FIG. 1 is a flowchart of the steps performed by prior art training approach methods.

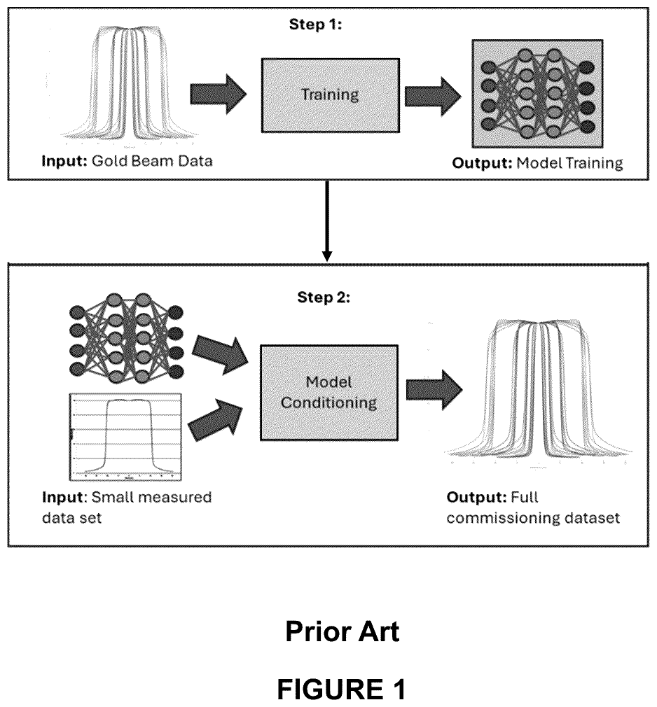

FIG. 2 is a flowchart of the steps performed by the training approach methods according to the invention.

FIG. 3 illustrates an exemplary computer system that may be used to implement the methods according to the invention; and

FIG. 4 illustrates a cloud-based system that may be used to implement the methods according to the invention.

### DETAILED DESCRIPTION

The invention provides a machine learning model to generate all necessary LINAC physics data needed from a very few physical measurements, therefore reducing the cost (e.g., labor intensiveness, machine down time, measurement errors, etc.). Specifically, a Wavelet-based Implicit Neural Network (WINN) is employed with a Morlet wavelet function. The model activated by Morlet wavelet, with its unique mathematical property of combining a cosine term filtered by a Gaussian envelope, consistently meets clinical tolerance with gamma passing rates over 95% and mean absolute errors ≤0.5%.

The overall behavior of signals through the Gaussian component and the spatially varying details through the cosine component is effectively captured. According to one embodiment, the model has a total size of less than 1 MB, making it ideal for deployment in resource-constrained clinical settings.

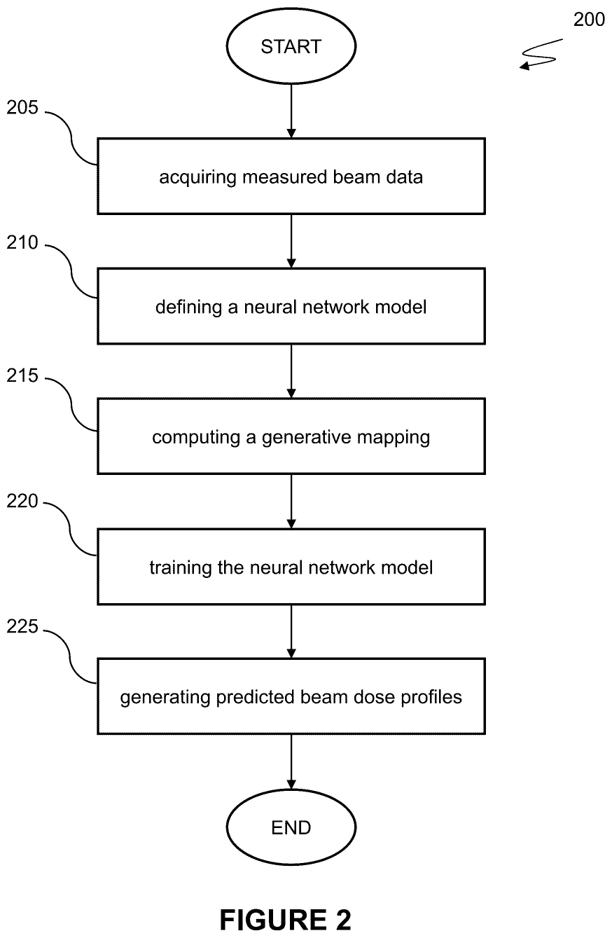

The invention is directed to a computer-implemented method for generating radiation beam dose profiles. FIG. 2 illustrates a flowchart 200 of the steps performed the training approach methods according to the invention. As shown at step 205 measured beam data is acquired. The measured beam data comprises spatial coordinates, field size indices, and corresponding measured dose values.

At step 210 a neural network model is defined. The neural network model comprises parameters and a wavelet activation function. The spatial coordinates are normalized to a predetermined spatial range and the dose values are normalized to a normalized dose range. According to one embodiment, the neural network model is a Wavelet-based Implicit Neural Network and the wavelet activation function is a Morlet wavelet activation function.

At step 215 a generative mapping is computed to predicted dose values. The generative mapping is computed from normalized spatial coordinates and field size indices

The neural network model is trained at step 220. The neural network model is trained by minimizing a loss function. Steps for training the neural network model comprise embedding prior knowledge by performing a first training stage using a first dataset of measured beam profiles across multiple field sizes, and conditioning the model by performing a second training stage using a single machine-specific measured beam profile to adapt the parameter to a linear accelerator.

Predicted beam dose profiles are generated for a linear accelerator at step 225. the predicted beam dose profiles for a linear accelerator are generated based on the conditioned neural network model.

Implicit neural representations (INRs) have recently gained popularity in deep learning, achieving state-of-the-art results in diverse tasks. Unlike conventional approaches, INRs don't explicitly rely on discrete representations such as voxel grids, meshes, or point clouds common in computer vision. Instead. INRs use continuous representation, implicitly encoding data as a generative function, supporting compact representation by using weights as codes to parametrize data features with a small memory footprint.

Generally, if v[1 . . . n] is an n-dimensional vector, an INR models v as a continuous function v(x), which is defined at x=1, 2, . . . , n, and interpolates the values for other x in a continuous domain. INRs have proven to be very effective with limited training data. For example, Dupont et al. demonstrated the effectiveness of INRs for image compression in COIN (Compression with Implicit Neural representation) and extended it to other modalities in COIN++ [24].

For commissioning LINAC beam profiles, INRs naturally handle continuous spatial inputs, predicting radiation dose dynamically and interpolating limited data for generalization.

### Wavelet Neural Networks are Examined.

The robustness of a neural network heavily depends on the choice of its nonlinearity, i.e., its activation functions. Some popular activations functions used for INRs are the sine function in SIREN and Gaussian activation functions. Wavelets' noise resistance, as compared to Gaussian and sine activation, suggests that replacing neurons with wavelons in a model should enhance its learning efficiency and reduce the model size.

A wavelon outputs a weighted sum of its components, defined as:

$$y = {\sum }_{j = 1}^{L} {\sum }_{i = 1}^{{n}_{j}} {w}_{ij} {Ψ}_{{a}_{ij} , {t}_{ij}} ( x ) + {b}_{ij} \qquad ( 1 )$$

where L is the total number of layers, nⱼ is the number of wavelons in layer j, and wᵢⱼ, bᵢⱼ, αᵢⱼ and tᵢⱼ are the weight, bias, dilation and translation parameters for the i-th wavelon in layer j. Wavelets have shown to be effective particularly for ill-posed problems that exhibit uncertainties, and minor positional shifts.

INR is combined with a Wavelet-based Implicit Neural Network (WINN) for radiation therapy commissioning application. WINN is utilized for LINAC commissioning and QA. LINAC beam profiles are viewed as a continuous, differentiable generator function:

$${ℳ}_{∅} : v \to d \qquad ( 2 )$$

where, v∈ [symbol] represents the location coordinates (x, y, z) and field size f_{S}. [symbol] is a generative function that encodes beam signals features within its parameters Ø. It maps spatial coordinates v to corresponding radiation beam dose d, minimizing the L1 loss function defined as:

$$ℒ = \frac{1}{N} {\sum }_{i = 1}^{N} {‖ {ℳ}_{∅} ( {v}_{i} ) - {d}_{i} ‖}_{1} \qquad ( 3 )$$

where N is the total number of samples, vᵢ is the i-th spatial coordinate, and di is the corresponding measured beam dose to vᵢ. The L1-norm, ∥·∥₁, sums the absolute differences between predicted and measured doses.

For data acquisition, physically measured beam data for commissioning Elekta LINACs from two totally unrelated radiation oncology centers is used, i.e., no shared clinical personal, no common commissioning and QA protocols, different dosimetry devices, etc. One dataset was used for embedding prior knowledge, while the second dataset was used for verification.

All beam data were collected with flattening filter (WFF) at 6 MV energy and 100 cm source-to-surface distance (SSD). The invention focuses on beam profiles predictions, which are most of the physics data and the most challenging part of the prediction model. During verification, the model was first conditioned with a single measured beam profile, before generating the rest of the profiles and evaluated against the actual measurements.

For implicit neural representation of the beam profiles, spatial coordinates (x, y, z) are normalized to [0,1] using the maximum beam scan range and depth. Field size is indexed from 0 to N−1 for N field sizes, and doses are normalized to [0,1] using the maximum dose per profile.

A two-stage training methodology is followed. In the first stage, prior knowledge from beam profiles across various field sizes is embedded into WINN's parameters (weights and biases) to enable accurate dose prediction at any spatial coordinate. Accuracy is validated using water tank measurements and Gamma analysis using a (1%/1 mm criterion).

The second stage conditions the model with a single machine-specific measured profile, addressing variations in design and assembly even among LINACs of the same model, vendor and configuration. After conditioning, a full set of dose profiles are generated for all field sizes, allowing dose queries at any position. This approach streamlines LINAC commissioning and QA by reducing measurement needs while ensuring high accuracy.

The wavelet activation function determined to be suitable for representing spatially variant beam data and validate their predictive performance under challenging conditions is the Morlet function. However, other wavelet activations are examined.

The Symmetric Gaussian Wavelet is obtained by modifying a Gaussian function to meet wavelet criteria by introducing translation and scale coefficients. The mathematical definition is given by:

$$ψ ( x ) = exp \frac{- {x}^{2}}{2} \qquad ( 4 )$$

The Mexican Hat Wavelet is derived from the second derivative of the Gaussian function. The mathematical definition is provided by:

$$ψ ( x ) = ( a - b {x}^{2} ) exp ( \frac{- {x}^{2}}{2} ) \qquad ( 5 )$$

The Asymmetric Gaussian Wavelet is Gaussian function, exhibiting asymmetric due to the linear term (−x), which introduces a skew in the wavelet. The mathematical definition is given by:

$$ψ ( x ) = - x \cdot exp ( \frac{- {x}^{2}}{2} ) \qquad ( 6 )$$

The Morlet Wavelet is a combination of a wave function and a Gaussian. The mathematical definition is provided by:

$$ψ ( x ) = cos ω x {exp}^{\frac{- {x}^{2}}{2}} \qquad ( 7 )$$

Wavelets activations in gradient-based optimization must be infinitely differentiable to ensure smooth, predictable output changes, and minimization of the loss in Equation (3).

In deep learning, initial layers capture core features, while deeper layers capture details. To emphasize primary patterns, neurons in deeper layers were reduced, anticipating further conditioning to adapt to specific LINACs. This design alleviates the complexity of the model and acts as regularization by focusing on essential features early and abstracting details later. The five-layers model progressively decreases neurons (1500, 1225, 950, 675, 400).

It is well known that in deep learning, increasing the model capacity, i.e., depth and width of the neural network can improve performance; however, it also raises computational complexity and can lead to issues such as overfitting, vanishing or exploding gradients.

In clinical practice, with constrained computational resources, real-time result requirements, it is essential to deploy small models that can be site-conditioned quickly. In another embodiment, the model is reduced from 5 to 3 layers, with 500, 275 and 50 neurons (an over 80% reduction in the number of neurons), and experimented with different activation functions.

The Morlet Wavelet performed comparably with a much smaller model, while other activation functions suffered an order of magnitude increase in errors. The superior performance of Morlet wavelet results from its mathematical characteristics. The Morlet Wavelet can be viewed as a combination between a wave and a Gaussian. A closer examination of the input dose profiles reveals that the profile has an outline like Gaussian while the sharp edges of the dose fall off requires a high-frequency wave function to capture its characteristics. The better performance of the Morlet wavelet activation function is due to its ability to capture the core features of the input with its Gaussian component and fine details with its wave component. The Morlet-activated WINN model consists of only 158 thousand parameters, achieving 99.46% reduction in model parameters. This results in a model size of less than 1 MB (approximately 0.633 MB), making it ideal for deployment on edge devices and embedded systems in clinical settings.

A wavelet neural network is used for its robustness under noise so that the model can be robust under setup errors. This is validated by injecting Gaussian noise into 1% of the beam profiles. This noise, with a mean of 0 and standard deviation of 0.01.

The invention provides a deep learning model based on the wavelet implicit neural network (WINN) for radiotherapy commissioning and quality assurance. The key to the WINN model is the use of Morlet wavelet activation functions, which reduces the model size. The wavelet model's noise resistance and its small size enables faster training and conditioning, making it highly suitable for deployment in resource-constrained environments.

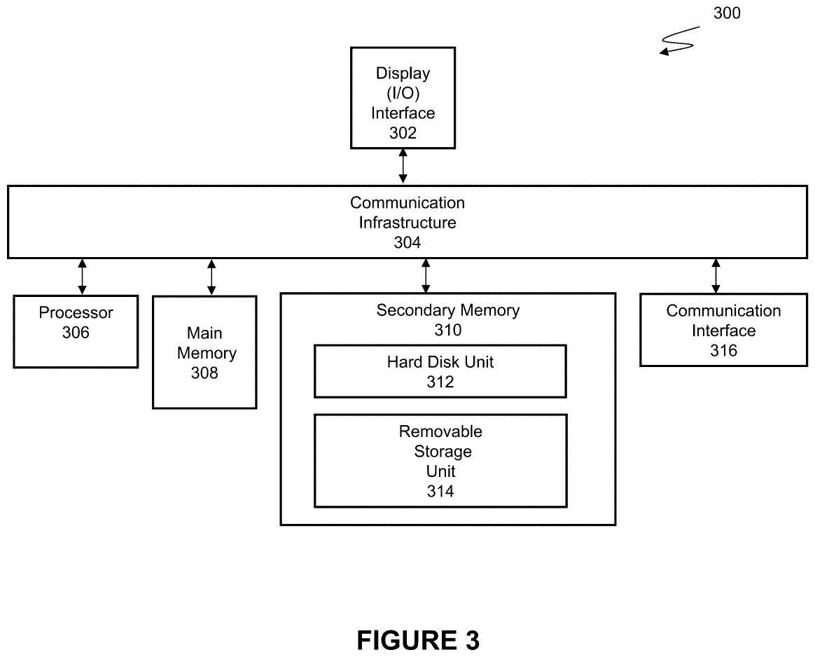

FIG. 3 illustrates an exemplary computer system 300 that may be used to implement the methods according to the invention. One or more computer systems 300 may carry out the methods presented herein as computer code.

Computer system 300 includes an input/output display interface 302 connected to communication infrastructure 304—such as a bus—which forwards data such as graphics, text, and information, from the communication infrastructure 304 or from a frame buffer (not shown) to other components of the computer system 300. The input/output display interface 302 may be, for example, a keyboard, touch screen, joystick, trackball, mouse, monitor, speaker, printer, any other computer peripheral device, or any combination thereof, capable of entering and/or viewing data.

Computer system 300 includes one or more processors 306, which may be a special purpose or a general-purpose digital signal processor that processes certain information. Computer system 300 also includes a main memory 308, for example random access memory (“RAM”), read-only memory (“ROM”), mass storage device, or any combination of tangible, non-transitory memory. Computer system 300 may also include a secondary memory 310 such as a hard disk unit 312, a removable storage unit 314, or any combination of tangible, non-transitory memory. Computer system 300 may also include a communication interface 316, for example, a modem, a network interface (such as an Ethernet card or Ethernet cable), a communication port, a PCMCIA slot and card, wired or wireless systems (such as Wi-Fi, Bluetooth, Infrared), local area networks, wide area networks, intranets, etc.

It is contemplated that the main memory 308, secondary memory 310, communication interface 316, or a combination thereof, function as a computer usable storage medium, otherwise referred to as a computer readable storage medium, to store and/or access computer software including computer instructions. For example, computer programs or other instructions may be loaded into the computer system 300 such as through a removable storage device, for example, a floppy disk, ZIP disks, magnetic tape, portable flash drive, optical disk such as a CD or DVD or Blu-ray, Micro-Electro-Mechanical Systems (“MEMS”), nanotechnological apparatus. Specifically, computer software including computer instructions may be transferred from the removable storage unit 314 or hard disc unit 312 to the secondary memory 310 or through the communication infrastructure 304 to the main memory 308 of the computer system 300.

Communication interface 316 allows software, instructions and data to be transferred between the computer system 300 and external devices or external networks. Software, instructions, and/or data transferred by the communication interface 316 are typically in the form of signals that may be electronic, electromagnetic, optical, or other signals capable of being sent and received by the communication interface 316. Signals may be sent and received using wire or cable, fiber optics, a phone line, a cellular phone link, a Radio Frequency (“RF”) link, wireless link, or other communication channels.

Computer programs, when executed, enable the computer system 300, particularly the processor 306, to implement the methods of the invention according to computer software including instructions.

The computer system 300 described herein may perform any one of, or any combination of, the steps of any of the methods presented herein. It is also contemplated that the methods according to the invention may be performed automatically or may be invoked by some form of manual intervention.

The computer system 300 of FIG. 3 is provided only for purposes of illustration, such that the invention is not limited to this specific embodiment. It is appreciated that a person skilled in the relevant art knows how to program and implement the invention using any computer system.

The computer system 300 may be a handheld device and include any small-sized computer device including, for example, a personal digital assistant (“PDA”), smart hand-held computing device, cellular telephone, or a laptop or netbook computer, hand held console or MP3 player, tablet, or similar hand held computer device

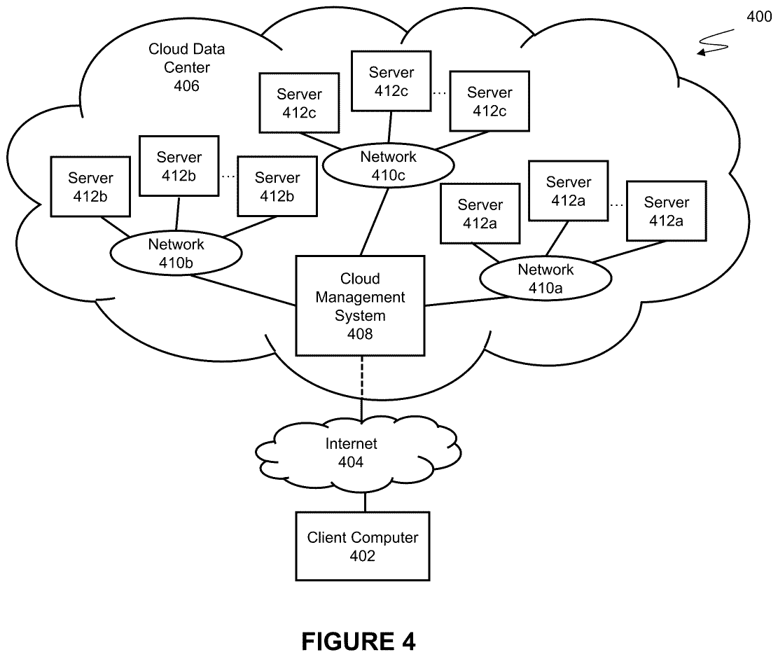

FIG. 4 illustrates an exemplary cloud computing system 400 that may be an embodiment of the invention. The cloud computing system 400 includes a plurality of interconnected computing environments. The cloud computing system 400 utilizes the resources from various networks as a collective virtual computer, where the services and applications can run independently from a particular computer or server configuration making hardware less important.

Specifically, the cloud computing system 400 includes at least one client computer 402. The client computer 402 may be any device through the use of which a distributed computing environment may be accessed to perform the methods disclosed herein, for example, a traditional computer, portable computer, mobile phone, personal digital assistant, tablet to name a few. The client computer 402 includes memory such as random access memory (RAM), read-only memory (ROM), mass storage device, or any combination thereof. The memory functions as a computer usable storage medium, otherwise referred to as a computer readable storage medium, to store and/or access computer software and/or instructions.

The client computer 402 also includes a communications interface, for example, a modem, a network interface (such as an Ethernet card), a communications port, a PCMCIA slot and card, wired or wireless systems, etc. The communications interface allows communication through transferred signals between the client computer 402 and external devices including networks such as the Internet 404 and cloud data center 406. Communication may be implemented using wireless or wired capability such as cable, fiber optics, a phone line, a cellular phone link, radio waves or other communication channels.

The client computer 402 establishes communication with the Internet 404—specifically to one or more servers—to, in turn, establish communication with one or more cloud data centers 406. A cloud data center 406 includes one or more networks 410a, 410b, 410c managed through a cloud management system 408. Each network 410a, 410b, 410c includes resource servers 412a, 412b, 412c, respectively. Servers 412a, 412b, 412c permit access to a collection of computing resources and components that can be invoked to instantiate a virtual machine, process, or other resource for a limited or defined duration. For example, one group of resource servers can host and serve an operating system or components thereof to deliver and instantiate a virtual machine. Another group of resource servers can accept requests to host computing cycles or processor time, to supply a defined level of processing power for a virtual machine. A further group of resource servers can host and serve applications to load on an instantiation of a virtual machine, such as an email client, a browser application, a messaging application, or other applications or software.

The cloud management system 408 can comprise a dedicated or centralized server and/or other software, hardware, and network tools to communicate with one or more networks 410a, 410b, 410c, such as the Internet or other public or private network, with all sets of resource servers 412a, 412b, 412c. The cloud management system 408 may be configured to query and identify the computing resources and components managed by the set of resource servers 412a, 412b, 412c needed and available for use in the cloud data center 406. Specifically, the cloud management system 408 may be configured to identify the hardware resources and components such as type and amount of processing power, type and amount of memory, type and amount of storage, type and amount of network bandwidth and the like, of the set of resource servers 412a, 412b, 412c needed and available for use in the cloud data center 406. Likewise, the cloud management system 408 can be configured to identify the software resources and components, such as type of Operating System (OS), application programs, and the like, of the set of resource servers 412a, 412b, 412c needed and available for use in the cloud data center 406.

The invention is also directed to computer products, otherwise referred to as computer program products, to provide software to the cloud computing system 400. Computer products store software on any computer useable medium, known now or in the future. Such software, when executed, may implement the methods according to certain embodiments of the invention. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, optical storage devices, Micro-Electro-Mechanical Systems (MEMS), nanotechnological storage device, etc.), and communication mediums (e.g., wired and wireless communications networks, local area networks, wide area networks, intranets, etc.). It is to be appreciated that the embodiments described herein may be implemented using software, hardware, firmware, or combinations thereof.

The cloud computing system 400 of FIG. 4 is provided only for purposes of illustration and does not limit the invention to this specific embodiment. It is appreciated that a person skilled in the relevant art knows how to program and implement the invention using any computer system or network architecture.

While the disclosure is susceptible to various modifications and alternative forms, specific exemplary embodiments of the invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

While the disclosure is susceptible to various modifications and alternative forms, specific exemplary embodiments of the invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

1. A computer-implemented method for generating radiation beam dose profiles, the method comprising instructions stored in one or more non-transitory mediums for performing the steps of:
acquiring measured beam data;
defining a neural network model comprising parameters and a wavelet activation function;
computing a generative mapping to predicted dose values;
training the neural network model; and
generating predicted beam dose profiles for a linear accelerator.

2. The computer-implemented method according to claim 1 wherein the measured beam data comprises spatial coordinates, field size indices, and corresponding measured dose values.

3. The computer-implemented method according to claim 2 further comprising the step of normalizing the spatial coordinates to a predetermined spatial range and normalizing the dose values to a normalized dose range.

4. The computer-implemented method according to claim 1 wherein the generative mapping is computed from normalized spatial coordinates and field size indices.

5. The computer-implemented method according to claim 1 wherein the wavelet activation function is a Morlet wavelet activation function.

6. The computer-implemented method according to claim 1 wherein the neural network model is trained by minimizing a loss function.

7. The computer-implemented method according to claim 1 wherein the neural network model is trained according to the steps of:
embedding prior knowledge by performing a first training stage using a first dataset of measured beam profiles across multiple field sizes; and
conditioning the model by performing a second training stage using a single machine-specific measured beam profile to adapt the parameter to a linear accelerator.

8. The computer-implemented method according to claim 7 wherein the predicted beam dose profiles for a linear accelerator are generated based on the conditioned neural network model.

9. The computer-implemented method according to claim 1 wherein the neural network model is a Wavelet-based Implicit Neural Network.
